(19) United States
(12) Patent Application Publication
(10) Pub. No.: US 2020/0000834 A1
(43) Pub. Date: Jan. 2, 2020

(54) POLYPHENOL GLYCOSIDE-CONTAINING COMPOSITION

(71) Applicant: SUNSTAR INC., Osaka (JP)

(72) Inventors: Kayo HARADA, Osaka (JP); Yoko SONO, Osaka (JP); Takashi KUSAKARI, Osaka (JP)

(73) Assignee: SUNSTAR INC., Osaka (JP)

(21) Appl. No.: 16/453,460

(22) Filed: Jun. 26, 2019

(30) Foreign Application Priority Data
Jun. 29, 2018 (JP) 2018-124618

(51) Int. Cl. A61K 31/7048; A23L 29/30

(52) U.S. Cl. CPC A61K 31/7048; A23L 29/30

(57) ABSTRACT

The present invention provides a method of efficiently promoting AMPK and/or ACC phosphorylation. Specifically, the present invention provides a composition comprising trifolin and astragalin, wherein the content mass ratio of trifolin and astragalin is 1:0.2 to 3.

### TECHNICAL FIELD

The present invention relates to a composition comprising polyphenol glycoside (in particular, trifolin and astragalin) etc.

### BACKGROUND ART

AMP-activated protein kinase (AMPK) is one of the serine/threonine kinases (serine/threonine phosphoenzymes) that are highly conserved in eukaryotic cells, and plays an important role as an intracellular energy sensor. AMP-activated protein kinase, for example, has an effect of promoting glucose uptake, glycolysis, oxidation of fatty acid β, etc., and an effect of inhibiting glycogen synthesis, glyconeogenesis, and fatty acid or cholesterol synthesis. Accordingly, controlling AMPK activation is important to prevent or treat various illnesses (e.g., diabetes, obesity, or cancer).

One of the physiological functions that lower blood sugar levels is a “metabolism function through uptake of blood sugar from blood vessels (blood) into skeletal muscle.” The following action is known as a mechanism of this function: “skeletal muscle contraction stimulates phosphorylation (activation) of intracellular AMP kinase, which causes translocation of glucose transporter 4 in the cytoplasm into the cell membrane, thus resulting in blood sugar uptake into skeletal muscle cells.” It has also been revealed that sugar uptake into skeletal muscle cells does not necessarily require skeletal muscle contraction, but is caused by activation of AMP kinase. Accordingly, even when exercise cannot contract skeletal muscle, the function of blood sugar uptake into skeletal muscle is potentially achievable just like exercise is performed. In recent years, while focusing on this point, several “attempts to reduce blood sugar levels by oral intake of a substance that activates AMP kinase” have been proposed (Patent Literature (PTL) 1 and Patent Literature (PTL) 2).

Phosphorylation of AMPK stimulates phosphorylation of ACC (acetyl-CoA carboxylase), which is the direct target of phosphorylation; accordingly, phosphorylation of ACC is also an index showing the degree of AMPK activation. ACC phosphorylation is considered to inactivate ACC and inhibit conversion of acetyl CoA into malonyl CoA, thus controlling subsequent lipid metabolism. Accordingly, it can be said that the more the phosphorylation of AMPK and ACC is promoted, the more the sugar uptake and lipid oxidation (dissimilation) are activated.

Thus, promotion of AMPK and ACC phosphorylation, especially in skeletal myocytes, is expected to reduce blood sugar levels and prevent and/or improve obesity.

### CITATION LIST

### Patent Literature

PTL 1: JP2010-37323A

PTL 2: JP2011-37732A

PTL 3: WO2015/033898

PTL 4: JP2014-198684A

### SUMMARY OF INVENTION

### Technical Problem

An object of the present invention is to provide a method of efficiently promoting AMPK and/or ACC phosphorylation.

### Solution to Problem

The present inventors found that the combination use of trifolin and astragalin in a specific mass ratio may attain significantly high AMPK and ACC phosphorylation promotion effects. The inventors conducted further modification, and accomplished the present invention.

The present invention includes the subject matter described in the following items.

1. A composition comprising trifolin and astragalin, wherein the content mass ratio of trifolin and astragalin is 1:0.2 to 3.
2. The composition according to Item 1, wherein the content mass ratio of trifolin and astragalin is 1:0.5 to 2.
3. The composition according to Item 1 or 2, further comprising hyperoside and/or isoquercitrin.
4. The composition according to any one of Items 1 to 3, wherein the composition is used for activating AMP-activated protein kinase and/or inactivating acetyl-CoA carboxylase.
5. The composition according to any one of Items 1 to 3 for reducing a blood sugar level.
6. The composition according to any one of Items 1 to 5 for use in foods, quasi-drugs, or drugs.

### Advantageous Effects of Invention

The present invention provides a technique of attaining excellent AMPK and ACC phosphorylation promotion effects.

### BRIEF DESCRIPTION OF DRAWINGS

FIG. 1 shows the results of western blotting in which the total amount of AMP kinase and ACC (acetyl-CoA carboxylase) and the amount of phosphorylated AMP kinase and ACC, each obtained by changing the combination amounts of trifolin and astragalin, are analyzed.

FIG. 2a shows the degree of the activation of AMP kinase obtained by changing the combination amounts of trifolin and astragalin.

FIG. 2b shows the degree of the phosphorylation of ACC (acetyl-CoA carboxylase) obtained by changing the combination amounts of trifolin and astragalin.

### DESCRIPTION OF EMBODIMENTS

The following describes embodiments of the present invention in more detail. The present invention preferably includes a specific composition as well as use and a production method of the composition; however, the present invention is not limited to these. The present invention encompasses everything disclosed in this specification and acknowledged by those skilled in the art.

The composition of the present invention comprises trifolin and astragalin, and the content mass ratio of trifolin and astragalin is 1:0.2 to 3. The composition is sometimes referred to as “composition of the present invention.”

As described above, the composition of the present invention comprises trifolin and astragalin in a mass ratio of 1:0.2 to 3. Trifolin and astragalin are polyphenol glycosides (specifically flavonoid glycosides), and more restrictively kaempferol glycosides. Trifolin is sometimes also referred to as kaempferol-3-O-galactoside (KGA). Astragalin (Astragalin) is sometimes also referred to as kaempferol-3-O-glucoside (KGU).

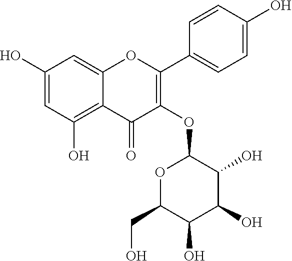 [SMILES: O=c1c(O[C@@H]2O[C@H](CO)[C@H](O)[C@H](O)[C@H]2O)c(-c2ccc(O)cc2)oc2cc(O)cc(O)c12]

Trifolin (kaempferol-3-O-galactoside)

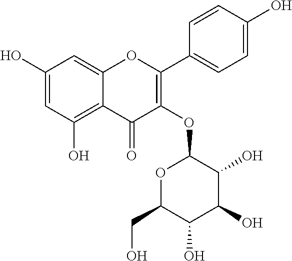 [SMILES: O=c1c(O[C@@H]2O[C@H](CO)[C@@H](O)[C@H](O)[C@H]2O)c(-c2ccc(O)cc2)oc2cc(O)cc(O)c12]

Astragalin (kaempferol-3-O-glucoside)

As described above, the content mass ratio of trifolin and astragalin (trifolin:astragalin) is 1:0.2 to 3. The upper limit or lower limit of the rate range may be 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, or 2.9. For example, the content mass ratio of trifolin and astragalin is more preferably 1:0.3 to 2.5, still more preferably 1:0.5 to 2, and even more preferably 1:1 to 2.

The composition of the present invention may further comprise another polyphenol glycoside. As another polyphenol glycoside, flavonoid glycosides are preferred, and quercetin glycosides are more preferred. Of these, hyperoside and/or isoquercitrin are preferably contained. Hyperoside is sometimes also referred to as quercetin-3-O-galactoside (QGA). Isoquercitrin is sometimes also referred to as quercetin-3-O-glucoside (QGU).

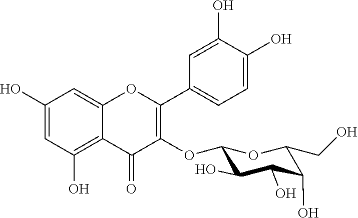

Hyperoside (quercetin-3-O-galactoside)

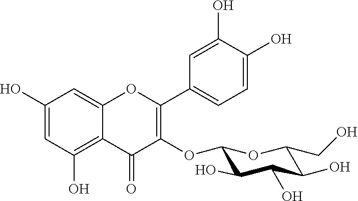

Isoquercitrin (quercetin-3-O-glucoside)

When the composition of the present invention comprises hyperoside and isoquercitrin, the content mass ratio of hyperoside and isoquercitrin (hyperoside:isoquercitrin) is preferably 1:0.2 to 3. The upper limit or lower limit of the rate range may be 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, or 2.9. For example, the content mass ratio of hyperoside and isoquercitrin is more preferably 1:0.3 to 2.8, still more preferably 1:0.5 to 2.5, and even more preferably 1:1 to 2.3.

Such polyphenol glycosides are known compounds and can be prepared by known methods or methods easily conceivable from the known methods. Additionally, commercially available products can be purchased and used. For example, products can be purchased from Extrasynthese S.A., Toctric Bio-technology, Sigma-Aldrich, Aobious Inc., EMMX Biotechnology LLC, Quality Phytochemicals LLC, Indofine Chemical Company, Inc., etc.

The composition of the present invention may further comprise other components. Examples of other components include pharmaceutically acceptable or food-hygiene-acceptable carriers. As such carriers, those known in the technical fields can be used. The content of trifolin and astragalin in the composition of the present invention is not particularly limited as long as the effects of the present invention are attained, and it is, for example, about 0.001 to 100 mass %, about 0.005 to 50 mass %, or about 0.01 to 30 mass %.

The composition of the present invention can be used as a food, drug, or quasi-drug. The injection method of the composition of the present invention is not particularly limited as long as the effects of the present invention are attained. The composition may be administered through the mouth, blood vessels, or skin. In particular, oral administration is preferred. Examples of foods include, in addition to general foods, functional foods or beverages, foods for patients, foods for specified health uses (FOSHU), foods with nutrient function claims (FNFC), dietary supplement foods, foods for exercise therapy, foods for slimming, etc.

The dosage form of the composition of the present invention is not particularly limited. When the composition is used as an oral composition, examples of the dosage form include hard capsules, soft capsules, supplements, chewable tablets, beverages, powder drinks, granules, films, and other forms. In addition, the composition can take other dosage forms, including beverages, such as tea beverages, sports drinks, cosmetic drinks, fruit juice beverages, carbonated beverages, liquors, soft drinks, jelly drinks, and concentrated beverages diluted with water, hot water, carbonated water, etc.; powder and granules that are drunk after being dissolved or suspended in water, hot water, etc.; dry solid forms, such as tablets; confectionery, such as tablet confectionery, jellies, snacks, baked goods, fried cakes, cakes, chocolate, gum, candies, and gummy candies; soup, noodles, rice, cereal, and other food forms. Among these, preferable forms for ordinary life include supplements, chewable tablets, one-shot drinks, etc.; and forms most preferable for intake for the purpose of promoting exercise effects include beverages, such as sports drinks. Further, these oral compositions can be provided to consumers in the form of packed foods that are placed in containers.

The composition of the present invention exhibits AMP kinase activation (i.e., phosphorylation promotion) effects and ACC (acetyl-CoA carboxylase) phosphorylation promotion (i.e., inactivation promotion) effects. Such effects are preferably attained especially in skeletal muscle cells. Having such a feature, the composition of the present invention can preferably attain, for example, glycometabolism improvement effects, particularly blood sugar level reduction effects. The composition of the present invention can be preferably used because of the above effects. In particular, regarding the ACC phosphorylation promotion (i.e., ACC inactivation promotion) effects, single use of trifolin attains almost no effects, but the use of trifolin and astragalin in a specific mass ratio attains particularly high effects.

The term “comprising” includes “consisting essentially of” and “consisting of.” The present invention entirely includes any combination of constituent elements explained in this specification.

Various characteristics (properties, structures, functions, etc.) detailed in the embodiments of the present invention may be combined in any manner to specify the subject matter of the present invention. In other words, the present invention includes all of the subject matter obtained by any combination of combinable characteristics described in this specification.

### EXAMPLES

The present invention is explained in detail below; however, it is not limited to the following Examples.

As shown in Table 1, trifolin (KGA) and/or astragalin (KGU) was added to a low-glucose DMEM medium and dissolved so that the total concentration of the trifolin (KGA) and/or astragalin (KGU) was 20 μM. The resultant was used in the following experiment. Since trifolin and astragalin have the same molecular weights, their concentration ratios are the same as the mass ratios. A medium without any addition was used as a negative control, and a medium containing AICAR (final concentration: 2 mM) was used as a positive control. AICAR is a substance that is metabolized to an analog of AMP when taken in a cell. AICAR is known as a substance significantly activating AMPK in skeletal muscle.

A mouse skeletal myotube cell line (C2C12) was inoculated in a 6-well cell culture plate. The cell line was cultured in a DMEM medium containing 10% fetal bovine serum and a 1% antibacterial agent at 37° C. in the presence of 5% carbon dioxide for 3 days. When the cells were grown to confluence, the medium was replaced with a DMEM medium containing 2% horse serum, and the cells were further cultured and differentiated into myotubers. The cells were then used for experiments. After the starvation of the cells in a serum-free medium for 3 hours, 1/10 of the amount of a medium containing trifolin and/or astragalin whose concentration had been adjusted to 10 times the final concentration was added, followed by treatment for 2 hours. After the cells were washed with PBS(−) twice, 80 μL of a cell lysis buffer containing a phosphatase inhibitor and a protease inhibitor was added, and each cell lycetate was collected with a cell scraper. After lysis of the cells, the supernatant was collected by centrifugation. The supernatant was stored at −80° C. until it was subjected to measurement. The protein concentration of each supernatant was measured. The protein concentration in each sample was adjusted to the same level. After the protein concentration of each supernatant was adjusted, a sample buffer (available from Thermo Scientific) was added, and the protein was heat-denatured. The resulting product was used in western blotting.

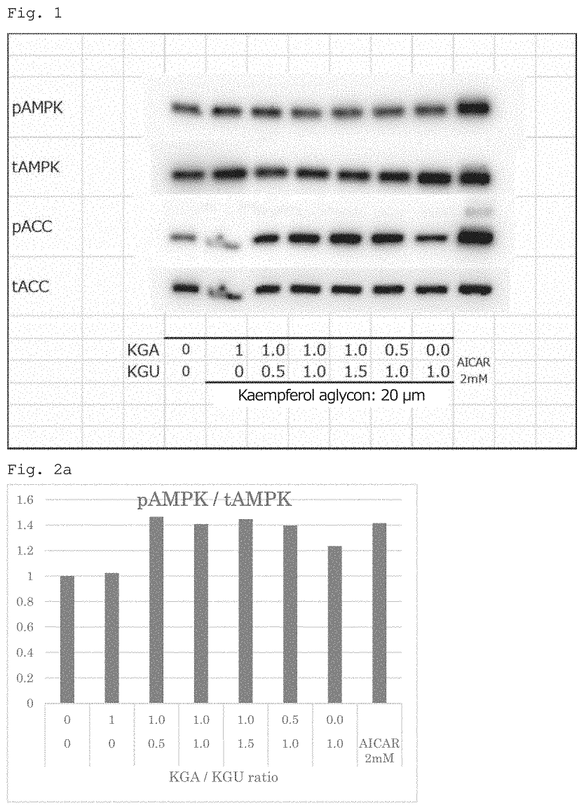

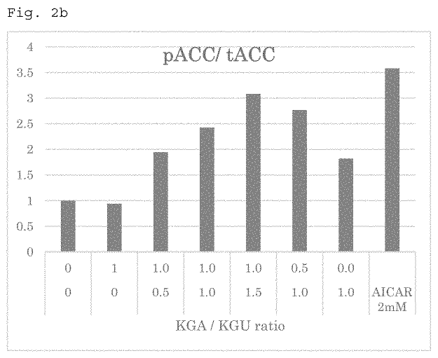

After SDS-PAGE, the protein was transferred to a PVDF membrane, and blocking was performed. Thereafter, as a primary antibody, an anti-phosphorylation AMPK antibody, an anti-total AMPKα antibody, an anti-phosphorylation ACC antibody, or an anti-total ACC antibody (all CST), each being hosted in a rabbit, was reacted. After sufficient washing, an HRP-labeled anti-rabbit secondary antibody was reacted. After sufficient washing, chemiluminescence (band) on the membrane was detected using a CCD camera imager (produced by GE Healthcare) (FIG. 1). The band intensity was quantified using image J (NIH). The degree of the activity (i.e., “phosphorylated AMPK/total AMPK (pAMPK/tAMPK)” or “phosphorylated ACC/total ACC (pACC/tACC)”) was expressed as a relative value with the value of the negative control being defined as 1 (FIG. 2a and FIG. 2b).

TABLE 1

| | | KGA | KGU |
|---|---|---|---|
| | | concentration | concentration |
| | KGA:KGU | (μM) | (μM) |
| KGA | 1.0:0 | 20 | 0 |
| KGA + KGU | 1.0:0.5 | 13.3 | 6.7 |
| KGA + KGU | 1.0:1.0 | 10 | 10 |
| KGA + KGU | 1.0:1.5 | 8 | 12 |
| KGA + KGU | 0.5:1.0 | 6.7 | 13.3 |
| KGU | 0:1.0 | 0 | 20 |

1. A composition comprising trifolin and astragalin, wherein the content mass ratio of trifolin and astragalin is 1:0.2 to 3.

2. The composition according to claim 1, wherein the content mass ratio of trifolin and astragalin is 1:0.5 to 2.

3. The composition according to claim 1, further comprising hyperoside and/or isoquercitrin.

4. The composition according to claim 1 for use in foods, quasi-drugs, or drugs.

5. A method of activating AMP-activated protein kinase in a skeletal muscle cell, comprising administering the composition according to claim 1 to a subject in need of sugar metabolism improvement.

6. A method of inactivating acetyl-CoA carboxylase in a skeletal muscle cell, comprising administering a required amount of the composition according to claim 1 to a subject in need of sugar metabolism improvement.

7. A method of reducing blood sugar levels, comprising administering a required amount of the composition according to claim 1 to a subject in need of reduction in blood sugar levels.

8. A method of reducing blood sugar levels, comprising administering a required amount of the composition according to claim 2 to a subject in need of reduction in blood sugar levels.

9. A method of reducing blood sugar levels, comprising administering a required amount of the composition according to claim 3 to a subject in need of reduction in blood sugar levels.
